(19) United States
(12) Patent Application Publication
(10) Pub. No.: US 2007/0048856 A1
(43) Pub. Date: Mar. 1, 2007

(54) GAS PURIFICATION APPARATUS AND PROCESS USING BIOFILTRATION AND ENZYMATIC REACTIONS

(76) Inventors: Carmen Parent, Quebec (CA); Richard Lepine, Ste-Foy (CA); Sylvie Fradette, Pintendre (CA); Alpha Barry, Ste-Foy (CA)

Correspondence Address: WARE FRESSOLA VAN DER SLUYS &;ADOLPHSON, LLP, BRADFORD GREEN, BUILDING 5, 755 MAIN STREET, P O BOX 224, MONROE, CT, 06468, US

(21) Appl. No.: 11/460,402

(22) Filed: Jul. 27, 2006

Related U.S. Application Data
(60) Provisional application No. 60/702,628, filed on Jul. 27, 2005.

(51) Int. Cl. A61L 9/01

(52) U.S. Cl. 435/266

(57) ABSTRACT

The invention provides a gas purification apparatus for purifying effluent gas by treating VOCs and the CO₂ gas, comprising a biofiltration chamber containing micro-organisms for degrading the VOCs and producing a partially cleansed gas containing CO₂ gas by-product; and an enzymatic chamber comprising an enzyme capable of catalyzing the hydration of the CO₂ gas by-product into bicarbonate ions and hydrogen ions, to produce an ion rich solution and a purified gas. Additionally, a gas purification process is provided.

### FIELD OF THE INVENTION

The present invention generally relates to industries involving effluent treatment, such as the forestry industry, and more particularly to a technology for purifying gas effluents with a mind to the conventions on climate change that have been developed with the Kyoto protocol. More specifically, the invention concerns a apparatus and process employing biofiltration and enzymatic reaction for achieving a more complete purification of gas effluents.

### BACKGROUND OF THE INVENTION

In the domain of processes for purifying gas effluents and in view of the conventions on climate change which have been developed with the Kyoto protocol, the treatment of industrial gas emissions cannot be overlooked or underestimated. Industrial gas effluents contain a variety of compounds, which vary in their toxicity, noxiousness, solubility and removability from gases. As the industrial standards in effluent gas purity increase in severity and subtlety, the known devices for purifying gas effluents are becoming antiquated and inadequate.

Certain effluent treatment processes and devices are known in the art to remove certain compounds from the effluent gas. The treatment of gas effluents by biofiltration, for instance, takes advantage of the natural degradation phenomenon of pollutants by micro-organisms for transforming different volatile organic compounds (VOCs) into degraded products that are much less harmful to the environment. Biofilters are present on the market and are used in many sectors of industry. In general, biofiltration processes include the degradation of polluting gases by micro-organisms that are typically fixed to a porous support (media). At the surface of this solid support, the micro-organisms grow in a thin layer called the biofilm. Polluted air flows within the biofilter, and the contaminants contained in the gaseous flow are absorbed by the biofilm where they are degraded by the micro-organisms. The concentration gradients of the pollutants between the gaseous phase and the biofilm cause a continuous transfer of the pollutants towards the biofilm. Biofilters are generally simple devices that use a combination of processes such as absorption, adsorption/desorption, and degradation of gaseous contaminants. U.S. patent application Ser. No. 10/475,842 (EGAN et al.), for example, describes a process and device for the biofiltration of VOCs.

The biostimulation of micro-organisms that have an affinity for specific volatile compounds is, however, not main stream. In addition, biofiltration processes give rise to various inefficiencies in the purification of effluent gases, which in turn may lead to such processes' failure in enabling a comprehensive treatment of the original effluent gases and in adhering to certain standards of gas purity. For example, biofiltration treatments fail to reduce the quantity of certain greenhouse gases targeted by the Kyoto protocol, in particular carbon dioxide (CO₂).

Another purification process known in the art uses enzymatic bioreaction technology, and has appeared under various forms. U.S. Pat. No. 6,524,843 (BLAIS et al.), for instance, describes certain embodiments of enzymatic bioreactors. The enzyme carbonic anhydrase has become a very useful tool in the management of greenhouse gases. Carbonic anhydrase (CA.EC 4.2.1.1.) is a metal enzyme containing zinc. This enzyme is capable of catalyzing a reversible hydration reaction of CO₂. The reaction is as follows:
CO₂+H₂⇄H⁺+HCO₃⁻

Its catalytic activity greatly accelerates the natural reaction that transforms CO₂ gas into bicarbonate ions and hydrogen ions. However, this enzymatic bioreaction technology suffers from certain disadvantages with regard to comprehensive purification of certain industrial effluent gases.

Of particular interest is the reduction of VOCs and CO₂ gas in industrial effluent gases.

With industrial processes producing effluent gases that contain diverse chemical compounds, and with the standards for comprehensive purification of gas effluents reaching new levels of rigorousness, there is a palpable and present need for a technology that enables a comprehensive treatment to manage diverse pollutants present in effluent gases.

### SUMMARY OF THE INVENTION

The present invention responds to the above-mentioned need by providing an apparatus and a process for purifying effluent gas.

Accordingly, the present invention provides a gas purification apparatus for purifying effluent gas containing volatile organic compounds (VOCs) by managing emissions of VOCs and carbon dioxide (CO₂). The apparatus includes a biofiltration chamber containing support media having disposed thereon micro-organisms suitable for performing degradation reactions of the VOCs and producing CO₂ gas by-product. The biofiltration chamber also has an inlet for receiving the effluent gas and an outlet for releasing a partially cleansed gas containing the CO₂ gas by-product. The apparatus also includes an enzymatic chamber having a gas inlet in fluid connection with the outlet of the biofiltration chamber for receiving therefrom the partially cleansed gas. The enzymatic chamber contains enzymes capable of catalyzing the hydration reaction of CO₂ into bicarbonate ions and hydrogen ions. The enzymatic chamber also has a liquid inlet for receiving a solvent to dissolve said CO₂ gas by-product, a gas outlet for releasing a purified gas and a liquid outlet for releasing an ion rich solution. The gas purification apparatus is thus able to reduce the amounts of VOCs and CO₂ by-product to produce the purified gas.

The present invention also provides a gas purification process for purifying effluent gas containing volatile organic compounds (VOCs) by managing emissions of VOCs and carbon dioxide (CO₂). The process includes the consecutive steps of:

degrading the VOCs contained in the effluent gas by biofiltration, thereby producing a partially cleansed gas containing CO₂ gas by-product;

dissolving the CO₂ gas by-product and catalyzing the hydration reaction of the CO₂ gas by-product into bicarbonate ions and hydrogen ions using enzymes, whereby the CO₂ gas by-product is removed from the partially cleansed gas; and

separating the bicarbonate and hydrogen ions from the partially cleansed gas to produce an ion rich solution and a purified gas.

### BRIEF DESCRIPTION OF THE DRAWINGS

The apparatus and process of gas purification by biofiltration and enzymatic reaction, according to certain embodiments of the present invention, are further described and illustrated in the drawings, in which:

FIG. 1 is a flow diagram showing the global apparatus used for experimentation of this new technology according to an embodiment of the present invention.

FIG. 2 is a flow diagram showing the internal functioning of a biofiltration chamber according to another embodiment of the invention.

FIG. 3 is a graph showing the concentration of CO₂ obtained at the entrance and at the exit of the enzymatic chamber following the treatment of the VOCs by the biofilter.

FIG. 4 is a flow diagram of the apparatus according to yet another embodiment of the present invention.

While the invention will be described in conjunction with example embodiments, it will be understood that it is not intended to limit the scope of the invention to such embodiments. On the contrary, it is intended to cover all alternatives, modifications and equivalents of the invention.

### DESCRIPTION OF THE PREFERRED EMBODIMENTS

The gas purification process and apparatus according to the present invention may be used in various industrial contexts. For instance, the purification apparatus may be used in the forestry/timber industry, where processes such as drying lignocellulosic fibres, high temperature pressing of composite wood panels and cooling of such panels after pressing, produce the effluent gas to be treated. Alternatively, the inventive apparatus or process may be used in other industries that produce complex effluent gases, for example, the pulp and paper industry, various chemical industries and the petroleum industry.

More specifically, the purification process and apparatus targets industrial effluent gases that contain varying amounts of volatile organic compounds (VOCs), which may include any number of chemicals—alcohols such as methanol, aldehydes such as formaldehyde, ketones, hydrocarbons such as methane and ethane, aromatic compounds such as benzene and toluene, and a myriad of other organic chemical species. By “VOC” it should be understood to include a broad range of chemical compounds that contain carbon atoms and may also contain oxygen, nitrogen, sulphur, halogens, hydrogen and other elements. VOCs are also “volatile” under certain pressure, temperature and concentration conditions such that the compounds may be degraded by micro-organisms. The volatility of these compounds, therefore, is more a matter of whether they may be absorbed into a biofilm, than whether they have enough vapour pressure to enter the atmosphere under STP conditions. Thus, many different compounds may be considered to be VOCs. For instance, the definition of VOCs found in the United States Environmental Protection Agency's Code of Federal Regulations, contains a plurality of concrete examples of VOCs, but may not contain an exhaustive list of VOCs as they pertain to the invention described herein. The effluent gas may also include greenhouse gases such as CO₂ and CO, among others, in undesirable quantities.

The Effluent Gas Purification Apparatus

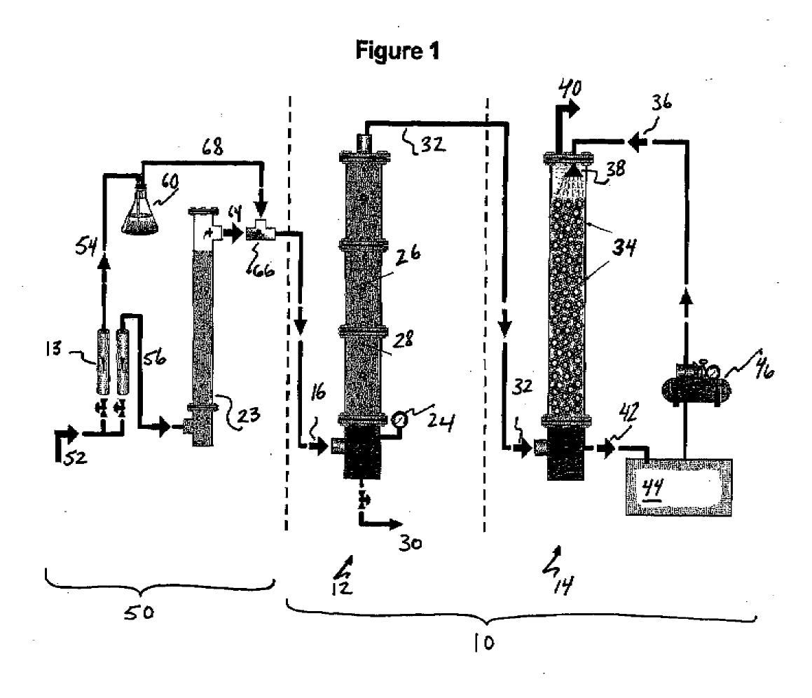

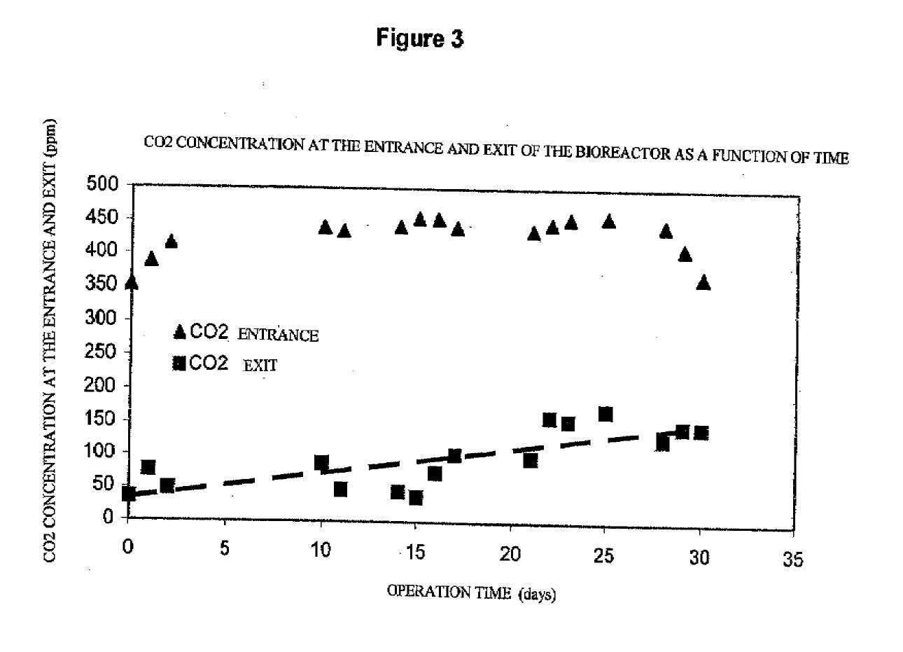

Preferred embodiments of the apparatus according to the present invention will be described hereafter in relation to FIGS. 1, 2 and 4, and examples will be presented in relation to FIG. 3.

Referring to FIG. 1, the effluent gas purification apparatus 10 includes at least two chambers for processing the effluent gas. The effluent gases containing VOCs are fed into the gas purification apparatus 10. In this preferred embodiment, the gas effluent is fed into a biofiltration chamber 12 and is subsequently fed into an enzymatic chamber 14. The biofiltration chamber 12 enables the degradation of the VOCs biologically, which generates CO₂ gas as a by-product. The enzymatic chamber 14 then reduces the amount of CO₂ in the effluent gas to produce a purified gas at its outlet. It will thus be appreciated that it is particularly advantageous to treat an effluent gas containing VOCs using the present invention, as the biofiltration and enzymatic chambers 12, 14 cooperate to purify the effluent gas with respect to VOCs as well as CO₂ gas. The close management of such noteworthy pollutants may, therefore, be performed and coordinated.

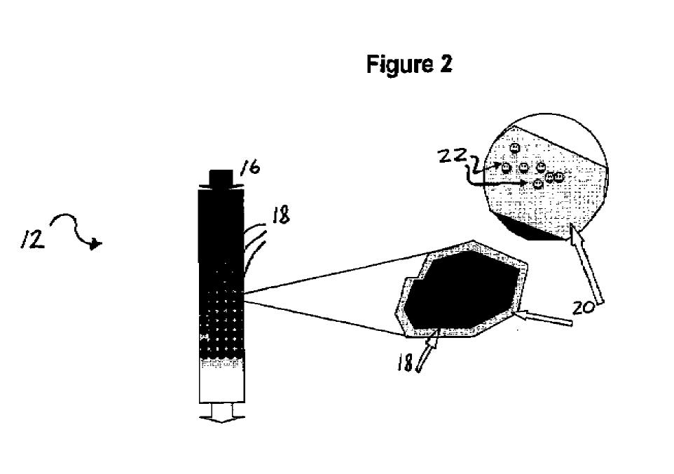

Referring still to FIG. 1, the gas purification apparatus 10 includes separate biofiltration and enzymatic chambers 12, 14. An effluent gas inlet stream 16 is provided for feeding the VOC-containing effluent into the apparatus 10. In this embodiment, the effluent gas is fed directly into the biofiltration unit 12. The biofiltration unit 12 is preferably in the form of a column, and the incoming effluent gas 16 is preferably fed to the bottom of the biofiltration column 12. Alternatively, and as illustrated in FIG. 2, the effluent gas may be fed to the top of the biofiltration column 12. The biofiltration chamber 12 may also take the form of a fluidized bed reaction chamber (not illustrated), a rotary reaction chamber (not illustrated) or another form suitable for enabling the biological degradation of VOCs.

Still referring to FIG. 2, the biofiltration column 12 contains support media 18. Preferably, the support media 18 are made of bark, but they may also include a number of appropriate supports or packings known in the art. Preferably, the support media 18 are porous, solid supports and may also be materials such as compost, peels, wood chips, synthetic materials, rocks, volcanic rocks or combinations of such materials. On the support media 18, a biofilm 20 is provided and includes one or a variety of micro-organisms 22. The micro-organisms 22 may be grown on the support media 18 using techniques known in the art. The effluent gas 16 flows into the biofiltration column 12 and is at least partially absorbed into the biofilm 20. The VOCs contained in the effluent gas 16, in particular, are absorbed into the biofilm 20 and are degraded by the micro-organisms 22. The VOCs are first oxidized by the micro-organisms 22 and then broken down to various reaction products. The operating conditions of the biofiltration column 12 are controlled to maintain biofilm 20, which implies monitoring the pressure, temperature and effluent gas flowrate, while providing the micro-organisms 22 with sufficient nourishment and ensuring that the adequate amount of VOCs are being removed from the effluent gas 16. The concentration gradients of the pollutants between the gaseous phase and the biofilm 20 preferably cause a continuous transfer of the pollutant towards the biofilm 20.

The support media 18 in the biofiltration column 12 are capable of supporting microbial growth, whether bacterial or fungal. The micro-organisms 22 may be fixed to or grown on the support media 18. In a preferred embodiment, the support media 18 are enriched with a variety of micro-organisms 22, characterised in that they have a vast and substantial catalytic potential. In the presence of a given compound in the effluent gas, such as one or more VOCs that may also be called a “substrate”, the micro-organisms 22 produce inductible enzymes capable of degrading the given compound. Following this induction phase, the micro-organisms 22 grow while catalyzing the transformation of the compound in question. The degradation of VOCs by micro-organisms is nevertheless complex and may involve a variety of mechanisms that are intracellular and intercellular, depending on the specific organism, operating conditions and VOCs involved. Degradation gases such as CO₂ gas, among others, are produced during this process. The degradation products—their production rate and composition—also depend on the specific VOC or combination of VOCs that are degraded by the micro-organisms 22.

The apparatus 10 preferably includes a single biofiltration chamber 12, which may have a variety of micro-organisms provided therein. Alternatively, there may be a plurality of biofiltration chambers (not illustrated), in series or in parallel, each provided with different micro-organism(s), to optimize the degradation. This latter arrangement is preferred when the conditions of chemical degradation require microbes with significantly different characteristics.

Referring to FIG. 1, it should be noted that the effluent gas 16 may be pre-treated before entering the gas purification apparatus 10, notably by a step of humidification in a humidifier column 23. Such a column 23 is preferable when the incoming effluent is dry enough to potentially hinder the growth of the micro-organisms 22 in the biofiltration chamber 12. Alternatively, the biofiltration chamber 12 may be humidified in another way, such as by adding water through a separate inlet stream (not illustrated) or according to techniques known in the art.

Referring still to FIG. 1, the biofiltration column 12 is advantageously provided with a pressure gauge 24 and various ports 26, 28 for taking measurements within the biofiltration column 12. A biomass sample port 26 is used to remove samples of biomass from the column to be tested and monitored. Gas sample ports 28 are used to remove samples of effluent gas along the column 12, to monitor the cleaning capacity, quality and progress in the biofilter column 12. Other ports for measuring conditions relating to the gas flow, temperature distribution and reaction progress are preferably present.

The biofiltration column 12 is also provided with an evacuation stream 30, through which condensation as well as liquid run-off are expelled from the biofilter 12. The biofiltration chamber 12 further includes a partially cleansed gas outlet stream 32, which preferably exits the top of the biofiltration column 12. The partially cleansed gas stream 32 has been subjected to biofiltration and therefore contains a reduced amount of VOCs. This partially cleansed gas stream 32 also contains greenhouse and other gases that were not able to be processed by the micro-organisms 22. Of particular interest is the elevated quantity of CO₂ gas in this clean gas stream 32, due to the CO₂ gas by-product generated in the biofiltration chamber 12 in addition to that contained in the original effluent gas 16. This elevated quantity of CO₂ gas has become a significant drawback of biofiltration, as its presence in expelled process gases is highly undesirable and, in many cases, unacceptable for greenhouse gas level standards. The partially cleansed gas stream 32 is thus adequately purified with respect to VOCs yet remains CO₂ rich, at least partly due to the latter's production in the biofiltration chamber 12.

Given the versatility of micro-organisms 22 with which the biofiltration chamber 12 may be stocked, the biofiltration chamber 12 is suited for metabolizing a large spectrum of substrates. Following the microbial induction of metabolic enzymes required for a specific substrate, the catalytic potential of the biofiltration chamber 12 attains a maximum in terms of degradation. A sharp rise in CO₂ gas, a commonly generated sub-product, is liberated and treated with the enzymatic chamber 14, as will be described in greater detail below.

Referring still to FIG. 1, once filtered and treated in the biofiltration chamber 12, the partially cleansed gas 32 is conducted with the degradation products to the enzymatic chamber 14. The partially cleansed gases 32 preferably enter the enzymatic chamber 14 at the bottom thereof. As illustrated, the enzymatic chamber 14 preferably takes the form of a packed tower chamber, containing enzymatic packing 34, partially or completely. The stream 32 passes through the enzymatic packing 34 present in the enzymatic column 14. The packing 34 contains at its surface an enzyme capable of transforming the dissolved CO₂ gas into bicarbonate and hydrogen ions in a hydration reaction.

In the case that the enzymatic chamber is a packed tower chamber, the packing 34 is preferably a solid packing, such as Rachig rings, “Tellerettes” or saddles. The packing 34 may be porous or non porous. The packing 34 is arranged to optimize the surface area to maximize the mass transfer. The enzyme is preferably covalently bound and immobilized to the surface of the packing 34. Alternatively, the enzymatic chamber may take the form of a fluidized bed reactor (not illustrated) in which the enzyme is in suspension, and into which the clean gas stream 32 from the biofiltration chamber 12 is fed using a bubbling unit (not illustrated) or another common means. The enzymatic chamber 14 may also take the form of another suitable reaction chamber known to a person skilled in the art and that may cooperate with the biofiltration chamber 12.

The enzyme is preferably covalently bound and immobilized on the packing 34, but may also be incorporated into the packing according to other techniques.

The enzyme used in the enzymatic chamber 14 is preferably carbonic anhydrase, which catalyses the hydration reaction of CO₂ gas. Alternatively, and particularly in the case where other undesirable gases were not degraded by the biofilter 12 or generated in the latter, other enzymes may be employed for purifying the gas stream 32. For instance, if the gas stream 32 contains high levels of carbon monoxide (CO) gas, then an enzyme geared to the dissolution or conversion of this gas may be preferably employed in the enzymatic chamber 14. The enzymatic chamber 14 is preferably provided with an enzyme with a catalytic potential for converting or degrading other by-product gases generated in the biofiltration chamber 12. The enzymatic chamber 14 may include a consortium of enzymes for simultaneously catalyzing different gases, or there may alternatively be a plurality of enzymatic chambers 14 in series provided with different enzymes for targeting different gases. For example, when carbon monoxide (CO) is present in the partially cleansed gas, an enzyme capable of catalyzing its degradation is preferably provided.

At the upper part of the lined enzymatic column 14, a solvent 36 is fed into the column 14 and preferably sprayed in micro droplets via a shower apparatus 38, thereby coating the enzymatic packing 34. Thus, in this preferred embodiment, the descendant liquid meets the entering gas counter-currently. There is therefore a mass transfer of CO₂ from the gas to the solvent 36. At least part of the CO₂ gas is dissolved in the solvent. This dissolving is a function of the temperature, pressure as well as the surface of interaction between the liquid phase and the gas phase. A purified gas stream 40 preferably exits the top of the enzymatic column 14 and may be released into the atmosphere.

It should also be noted that the enzymatic chamber 14 reduces the amount of CO₂ gas by-product generated in the biofiltration chamber 12, and, in the case where the raw effluent 16 contains CO₂ gas as well as VOCs, the enzymatic chamber 12 may also reduce the overall amount of CO₂ gas to produce the purified gas 40.

An ion-rich solvent 42, which is produced by the dissolving CO₂ gas into bicarbonate and hydrogen ions, preferably exits the bottom of the enzymatic reaction column 14 and is brought to a collection reservoir 44. From this collection reservoir 44, the ion-rich solvent 42 is preferably brought to a capture system (not illustrated), which can be an ionic exchanger type system or a precipitation system. The ions may be implicated in reactions with cations such as calcium and/or magnesium, etc, to improve the status of the industrial waste. The result is that the products of the transformation of CO₂ are captured, and thus eliminated. The solvent may thus be regenerated and may be recycled into the top of the enzymatic reaction column 14. The ion-rich solvent 42 may be brought to the capture system prior to being fed into the collection reservoir 44. As illustrated, a circulation pump 46 recycles at least part of the solvent from the collection reservoir 44 back into the enzymatic reaction column 14.

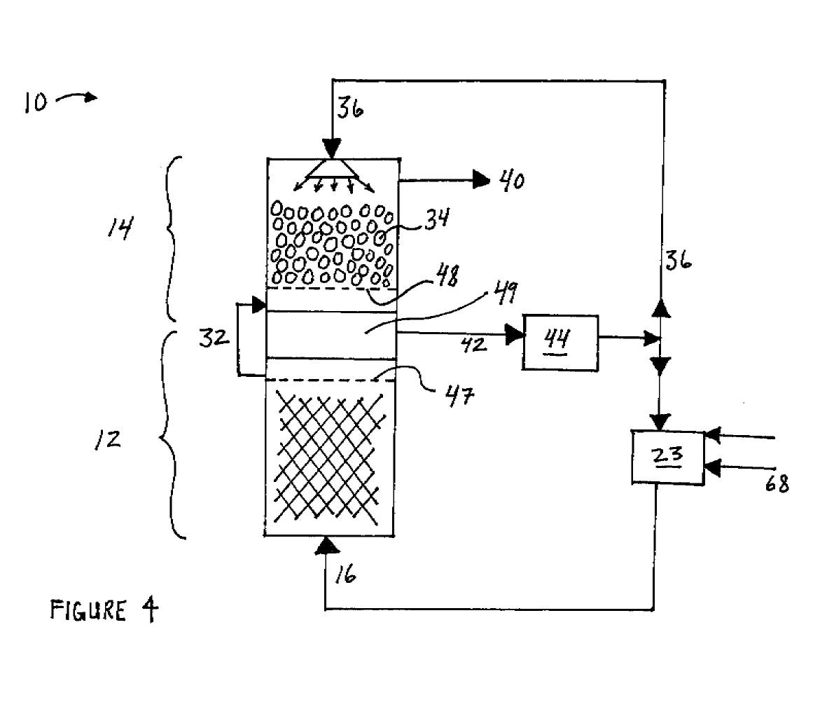

Referring now to FIG. 4, which represents another embodiment of the present invention, the biofiltration and enzymatic chambers 12, 14 are arranged as part of a reactor unit. In this embodiment, the biofiltration chamber 12 is provided with the effluent gas inlet stream 16 and the effluent gas passes through the support media 18. At the upper portion of the biofiltration chamber, there is provided a collection plate 47 which is perforated to allow the partially cleansed gas 32 to be gathered to then enter the enzymatic chamber 14. The partially cleansed gas 32 is preferably fed into the enzymatic chamber via a stream line, but may alternatively pass by other openings or conduits between the two chambers 12, 14.

The enzymatic chamber 14 receives the incoming partially cleansed gas 32 preferably at a bottom part thereof and by means of a distributor plate 48. The distributor plate 48 enables the incoming gas to be well distributed and diffused among the packing 34 within the enzymatic chamber 14. The solvent 36 and the enzymes enable the CO₂ by-product in the gas to be dissolved and converted into hydrogen and bicarbonate ions. The apparatus 10 also is able to withdraw the rich ion solution from the enzymatic chamber 12. Preferably, there is an collection tank 49 arranged to collect the used solvent, which has become an ion rich solution.

Preferably, the ion rich solution is employed to increase the efficiency of the apparatus or to benefit from the ions in a variety of ways. For instance, the ion rich solution may be regenerated using a variety of systems, such as an ion exchanger type system (not illustrated) or a precipitation system (not illustrated). The regenerated solvent may then be recycled back to the enzymatic chamber 14.

Furthermore, the ion rich solution may be converted using conversion means into a buffer solution to be added in desired quantities to the effluent gas 16 or to the biofiltration chamber 12 itself. Some micro-organisms may be sensitive to low pH levels and such a buffer solution may thus facilitate prolific and advantageous microbial growth. The buffer solution may be made in a number of ways using a number of conversion means. For example, the solvent may contain an amine-compound, which reacts with the hydrogen ions to remove them from solution to thereby raise the pH of the solution. In this case, therefore, the ion rich solution maintains a modestly basic pH, and thus may be provided directly to the effluent gas 16 just prior to its entering into the biofiltration chamber 12, or to the effluent gas in a pre-treatment. The bicarbonate ion-based buffer solution may be quite useful in promoting certain types of microbial growth. Alternatively, when the solvent does not contain compounds that react readily with the hydrogen ions, the ion rich solution is acidic and may be subjected to treatments to convert it to a basic buffer solution. Additions of compounds such as amines or appropriate bases, or elements such as calcium and the like in an appropriate form, are among the conversion means that may be employed to convert the ion rich solution into a buffer solution.

It may also be desirable to selectively use the ion rich solution for both recycling and buffering. Especially when the incoming effluent gas 16 fluctuates in composition, it is preferable to control the amount of buffering solution used for the biofiltration chamber 12. When the VOCs include acidic compounds, such as acetic acid for example, it may be particularly preferred to use the buffer solution.

A particular advantage of the apparatus 10 is that it enables a comprehensive management of VOCs and their degradation products, found in effluent gases. The apparatus 10 not only metabolizes VOCs via digestion by micro-organisms of a full range of substrates, and enables the capture of CO₂, but also assures the management of the CO₂ gas by-product emitted by the biofiltration chamber 12.

The Gas Purification Process

The gas purification process may be appreciated by referring to FIG. 1 and the following description.

The gas purification process for purifying effluent gas 16 containing volatile organic compounds (VOCs) allows the management of emissions of VOCs and carbon dioxide (CO₂). The process includes the consecutive steps of:

degrading the VOCs contained in the effluent gas by biofiltration, thereby producing a partially cleansed gas containing CO₂ gas by-product;

dissolving the CO₂ gas by-product and catalyzing the hydration reaction of the CO₂ gas by-product into bicarbonate ions and hydrogen ions using enzymes, whereby the CO₂ gas by-product is removed from the partially cleansed gas; and

separating the bicarbonate and hydrogen ions from the partially cleansed gas to produce an ion rich solution and a purified gas.

Preferably, the step of degrading the VOCs is performed in the biofiltration chamber 12 as illustrated in FIG. 1, 2 or 4. The step of dissolving the CO₂ gas by-product and catalyzing the hydration reaction is preferably performed in the enzymatic chamber 14 as illustrated in FIG. 1 or 4. The step of separating the ions from the purified gas 40 also preferably occurs in the enzymatic chamber 14. Alternatively, arrangements other than the preferred embodiments illustrated in FIGS. 1, 2 and 3 may be used to perform the process of the present invention.

Experimental Pilot Apparatus

Referring to FIG. 1, an experimental pilot apparatus was constructed to perform experimentation regarding this new technology. The pollutant effluent gas 16 entering the purification apparatus 10 was simulated by an effluent simulation unit 50. Effluent gases were simulated in the effluent simulating unit 50, wherein compressed air 52 was fed to the simulation unit 50 and split into first 54 and second 56 streams. Valves 58 were provided for regulating the proportion of compressed air in each stream. The first stream 54 was percolated through a VOC simulator 60. The second stream 56 was fed into the bottom of a humidifier column 62. A humidified air stream 64 exited the top of the humidifier column 62 and fed into a mixing zone 66 along with the VOC-containing gas 68 from the VOC simulator 60. The mixing of streams 64 and 68 produced the simulated effluent gas stream 16, which was fed into the bottom of the biofiltration chamber 12.

### EXAMPLES

Trials with this experimental pilot apparatus were performed to degrade VOCs issuing from the timber industry, such as formaldehyde, methanol, alpha-pinene and hexanal. Different degrees of oxidation of these products were observed. An increased amount of CO₂ was observed at the entrance of the enzymatic chamber 14 when components of this nature were introduced into the biofiltration chamber 12. The absence of the contaminants resulted in a decrease in CO₂ at the entrance of the enzymatic chamber 14. The results of this trial are represented hereafter. Different mixtures of volatile organic components were used in order to simulate different concentrations of the principle VOCs produced by the wood/timber industry. Drying of the lignocellulosic fiber, high temperature pressing of composite wood panels and cooling of these panels at the exit of the pressing are the main sources of these pollutants. Different mixtures were tested over four weeks of operation.

TABLE 1
Mixture of volatile organic compounds used

| Schedule in Days | Tested Mixture |
|---|---|
| 0 to 14 | No. 600 |
| 15 to 16 | No. 900 |
| 17 to 21 | No. 300 |
| 22 | No. 900 |
| 23 to 28 | No. 600 |
| 29 to 30 | No. 0 |

Mixture No. 300 had 300 ppm methanol at the entrance of the biofiltration chamber, whereas mixture Nos. 600 and 900 had 600 ppm and 900 ppm of methanol respectively. The mixtures also had, at most, approximately 60 ppm formaldehyde, 250 ppm acetic acid, 150 ppm phenol, 125 ppm hexanal, 50 ppm alpha-pinene at the entrance of the biofiltration chamber.

During 30 days, four mixtures were tested alternately. Each of these mixtures contained variable concentrations of the above-mentioned VOCs. The average percents of degradation for some specific VOCs are presented in Table 2.

TABLE 2
Rate of degradation of volatile compounds

| | Concentration at the exit | | |
|---|---|---|---|
| Volatile compounds | of the biofiltration chamber | | Percent of degradation |
| Formaldehyde | 4.5 to 9.5 | ppm | 91 to 100% |
| Methanol | 350 to 1250 | ppm | 30 to 96% |
| Alpha-pinene | 0.2 to 0.75 | ppm | 30 to 80% |
| Hexanal | 0.1 to 0.9 | ppm | 95 to 99% |

The average carbon dioxide concentration measured at the exit of the biofiltration chamber was 428 ppm±32 ppm. The mixture No. 0, containing no organic compounds, was used during the two last days of the experimental phase and a decrease in the concentration of carbon dioxide at the exit of the biofiltration chamber was observed. This also translated into a decrease in the performance of the enzymatic chamber since the performance of the enzymatic chamber is a function of the concentration of CO₂ present in the apparatus.

In conclusion, the apparatus and process have great potential, since they are able to transform VOCs, conforming not only to the norms relating to such rejected effluent gases but also to the policies put in place in accordance with the Kyoto protocol.

While one preferred embodiment of the invention was described above and illustrated in the Figs, the invention is not limited to these embodiments and many modifications may be made by a person skilled in the art while staying within the scope of what the inventor actually invented.

1. A gas purification apparatus for purifying effluent gas containing volatile organic compounds (VOCs) by managing emissions of VOCs and carbon dioxide (CO₂), comprising:
a biofiltration chamber containing support media having disposed thereon micro-organisms suitable for performing degradation reactions of the VOCs and producing CO₂ gas by-product, the biofiltration chamber having an inlet for receiving the effluent gas and an outlet for releasing a partially cleansed gas containing the CO₂ gas by-product; and
an enzymatic chamber having a gas inlet in fluid connection with the outlet of the biofiltration chamber for receiving therefrom the partially cleansed gas, said enzymatic chamber containing enzymes capable of catalyzing the hydration reaction of CO₂ into bicarbonate ions and hydrogen ions, said enzymatic chamber having also a liquid inlet for receiving a solvent to dissolve said CO₂ gas by-product, a gas outlet for releasing a purified gas and a liquid outlet for releasing an ion rich solution;
whereby the gas purification apparatus reduces the amounts of VOCs and CO₂ by-product to produce the purified gas.

2. The gas purification apparatus of claim 1, wherein the inlet of the biofiltration chamber is provided at a bottom part thereof and the outlet of the biofiltration chamber is provided at a top part thereof.

3. The gas purification apparatus of claim 2, wherein the biofiltration chamber has a liquid outlet for releasing condensation and liquid run-off.

4. The gas purification apparatus of claim 1, wherein the gas inlet of the enzymatic chamber is provided at a bottom part thereof and the liquid inlet is provided at a top part thereof, thereby enabling the partially cleansed gas and the solvent to flow counter-currently with respect to each other.

5. The gas purification apparatus of claim 1, further comprising a collection reservoir in fluid connection with the liquid outlet of the enzymatic chamber for collecting the ion rich solution.

6. The gas purification apparatus of claim 1, further comprising a regeneration unit having an inlet for receiving the ion rich solution, the regeneration unit regenerating the ion rich solution to produce a regenerated solvent, the regeneration unit also having an outlet in fluid connection with the liquid inlet of the enzymatic chamber for recycling the solvent thereto.

7. The gas purification apparatus of claim 1, further comprising conversion means for converting the ion rich solution into a buffer solution for the biofiltration chamber.

8. The gas purification apparatus of claim 1, wherein the support media of the micro-organisms are chosen from the group consisting of bark, peels, wood chips, synthetic packing, rocks and combinations thereof.

9. The gas purification apparatus of claim 1, wherein a plurality of different micro-organisms are provided on the support media for degrading a variety of VOCs.

10. The gas purification apparatus of claim 9, wherein the plurality of different micro-organisms comprise bacteria and/or fungi.

11. The gas purification apparatus of claim 1, wherein the enzymatic chamber is provided with a packing on which the enzymes are immobilized.

12. The gas purification apparatus of claim 11, wherein the enzymes include carbonic anhydrase.

13. A gas purification process for purifying effluent gas containing volatile organic compounds (VOCs) by managing emissions of VOCs and carbon dioxide (CO₂), the process comprising the consecutive steps of:
degrading the VOCs contained in the effluent gas by biofiltration, thereby producing a partially cleansed gas containing CO₂ gas by-product;
dissolving the CO₂ gas by-product and catalyzing the hydration reaction of the CO₂ gas by-product into bicarbonate ions and hydrogen ions using enzymes, whereby the CO₂ gas by-product is removed from the partially cleansed gas; and
separating the bicarbonate and hydrogen ions from the partially cleansed gas to produce an ion rich solution and a purified gas.

14. The gas purification apparatus of claim 13, comprising the additional steps of:
regenerating the ion rich solution by at least partially removing the hydrogen and bicarbonate ions therefrom to produce a regenerated solvent; and
recycling the regenerated solvent to dissolve the CO₂ gas by-product.

15. The gas purification apparatus of claim 14, wherein the step of regenerating the ion rich solution includes at least one of the following steps:
performing an ionic exchange; and
precipitating the bicarbonate and hydrogen ions.

16. The gas purification apparatus of claim 13, comprising the additional steps of:
converting at least part of the ion rich solution into a buffer solution; and
providing the buffer solution in the step of degrading the VOCs by biofiltration to thereby buffer said biofiltration.
